(19) United States
(12) Patent Application Publication
(10) Pub. No.: US 2011/0197320 A1
(43) Pub. Date: Aug. 11, 2011

(54) DYE COMPOSITION FOR FLOWER PETAL, DYEING METHOD FOR SAME, AND PLANT FURNISHED WITH PETALS DYED THEREBY

(76) Inventor: Hyeong Il Kweon, Gyeonggi-do (KR)

(21) Appl. No.: 13/123,526

(22) PCT Filed: Oct. 9, 2009
(86) PCT No.: PCT/KR2009/005772

§ 371 Date: Apr. 9, 2011

(30) Foreign Application Priority Data
Oct. 9, 2008 (KR) 10-2008-0099037

(51) Int. Cl. A01H 5/00; A01H 3/04; C09B 67/22

(52) U.S. Cl. 800/323; 8/499; 8/550; 8/557; 8/552

(57) ABSTRACT

The present invention relates to a dye composition for flower petals that dyes various colors seldom found in nature onto various plants including live flowers, or carries out noctilucence or photoluminescence treatment on the colors of live flowers as is to afford an opportunity to appreciate new beauty aesthetically and even in dark places so that the added value of merchandise may be further enhanced, and enables preservation over a long period as the petals do not wilt easily and also carry out noctilucence, photoluminescence, and dyeing treatment of flow petals easily and quickly stressing natural colors of live flower regardless of conditions and environments anywhere, at any time, and by any one, and with the dyeing method for same and a plant furnished with petals dyed thereby. The method of dyeing the petals of a plant comprises a materials preparation step wherein oil soluble dyes, pigment/paint, and a solubilization substance that mutually dissolves or mixes said pigment/paint are prepared as materials for dyeing, a dyeing chemical preparation step wherein said oil soluble dye and said pigment/paint prepared in a certain mixing ratio are dissolved or mixed using said solubilization material to prepare a dyeing solution; a dyeing solution spray step wherein said dyeing solution is placed in a certain holder, and said dyeing solution is sprayed onto the petals of a plant via said holder; and a dye solution drying step wherein the dyeing solution is dried.

### TECHNICAL FIELD

The present invention relates to a dye composition for flower petals of a plant, a dyeing method for the same, and a plant furnished with petals dyed thereby, and in particular to a dye composition for flower petals of a plant, a dyeing method for the same, and a plant furnished with petals dyed thereby which make it possible to enhance the added values of products in such a manner that people can have an opportunity to enjoy beauty even in a dark place by dyeing various colors, which cannot be easily found in nature, on various plants such as a real flower or something, or by letting the inherent colors of real flower have noctilucence and photoluminescence effects, and flower petals cannot easily wilt, thus preserving the same for a long time, and any person can easily and quickly dye flower petals regardless of surrounding conditions or environments and can treat the same to have noctilucence and photoluminescence effects while maintaining the inherent natural colors of real flowers.

### BACKGROUND ART

Various real flowers such as rose, common gypsophila and small flowered chrysanthemum and other plants inherently have limited colors, which might make customers who want various visual effects, feel boring.

It is why a lot of efforts are undergone to give leaves of plants or flowers or buds (hereinafter referred to flower petals) various manmade colors.

As one example, a desired color is dyed on petals using a dye in a conventional art or a dye is directly inputted into petals, thus making the petals have various colors.

When the petals are dyed by the above method, the dyed petals faster becomes wilt as compared the petals are dyed in nature, leaving the petals dirty. When touching the wilted petals, the dyes might smear the hands that have touched the dyed petals.

As described in the above art, a lot of time is needed to dye the petals along with many environmental limits.

For example, when the above method is directly applied, about two days generally take to dye the petals even when weather is clear.

When it is cloudy or there is a lot of moisture in the air, the transpiration operation of plants becomes much slower than usual time, thus requiring more than two days, and even when more times are given, it is hard to obtain a desired dyeing effect.

So, it is urgently needed to develop a dye composition for flower petal of a plant, a dyeing method for the same, and a plant furnished with petals dyed thereby which make it possible to enhance the added values of products in such a manner that people can have an opportunity to enjoy beauty even in a dark place by dyeing various colors, which cannot be easily found in nature, on various plants such as a real flower or something, or by letting the inherent colors of real flower have noctilucence and photoluminescence effects, and flower petals cannot easily wilt, thus preserving the same for a long time, and any person can easily and quickly dye flower petals regardless of surrounding conditions or environments and can treat the same to have noctilucence and photoluminescence effects while maintaining the inherent natural colors of real flowers.

### DISCLOSURE OF INVENTION

Accordingly, it is an object of the present invention to provide a dye composition for flower petal of a plant, a dyeing method for the same, and a plant furnished with petals dyed thereby which make it possible to enhance the added values of products in such a manner that people can have an opportunity to enjoy beauty even in a dark place by dyeing various colors, which cannot be easily found in nature, on various plants such as a real flower or something, or by letting the inherent colors of real flower have noctilucence and photoluminescence effects, and flower petals cannot easily wilt, thus preserving the same for a long time, and any person can easily and quickly dye flower petals regardless of surrounding conditions or environments and can treat the same to have noctilucence and photoluminescence effects while maintaining the inherent natural colors of real flowers.

To achieve the above objects, there is provided a method for dyeing the petals of plant, comprising a material preparation step for preparing oil soluble dye as dyeing material, pigment/pant and a dissolving substance for dissolving or mixing the oil soluble dye and the pigment/paint; a dyeing chemical preparation step for preparing a dyeing chemical by dissolving or mixing the oil soluble dye and the pigment/paint which have been prepared at a certain mixing ratio; a dyeing chemical spraying step for filling the dyeing chemical into a certain sprayer and spraying the dyeing chemical onto the petals of a plant via the sprayer; and a dyeing chemical drying step for drying the dyeing chemical following the dyeing chemical spraying step.

Here, the oil soluble dye is selected from common dyes except for edible dyes, and the pigment/paint is at least one selected from the group consisting of a functional pigment/paint containing chromalusion color pigment/paint, basecoat paint, thermochromic pigment/paint, photochromic pigment/paint, and photoluminescence pigment/paint, and a common pigment/paint, and the dissolving substance is selected from the group consisting of alcohol, acetone, and MEK which all have high evaporation performance.

The certain mixing ratio is 5 to 20 grams per 1 to 2 liters of the dissolving substance in case of the oil soluble transparent dye, and is 10 to 110 grams in case of the pigment/paint.

The dyeing chemical preparing step includes a sub-step for further adding a certain adhesive.

The adhesive is one selected from the group consisting of polyvinyl acetate resin solution, enamel and urethane resin adhesive, and the adhesive is mixed at a ratio of 10 to 20 weight % with respect to 100 weight % of the solution mixed with the oil soluble dye, the pigment/paint and the dissolving substance in the course of preparing dyeing chemicals.

To achieve the above objects, there is provided a plant characterized in that the plant is furnished with the petals dyed by the method of dyeing.

To achieve the above objects, there is provided a plant petal dyeing composition characterized in that the composition is prepared by dissolving or mixing 5 to 20 grams of oil soluble transparent dye and 10 to 110 grams of pigment/paint per 1 to 2 liters of the dissolving substance as a composition for dyeing the petals of plant.

Here, the oil soluble dye is selected from common dyes except for edible dyes, and the pigment/paint is at least one selected from the group consisting of a functional pigment/paint containing chromalusion color pigment/paint, basecoat paint, thermochromic pigment/paint, photochromic pigment/paint, and photoluminescence pigment/paint, and a common pigment/paint, and the dissolving substance is selected from the group consisting of alcohol, acetone, and MEK which all have high evaporation performance.

There is further provided an adhesive which is mixed at a ratio of 10 to 20 weight % with respect to 100 weight of the solution mixed with the oil soluble dye, the pigment/paint and the dissolving substance, and the adhesive is at least one selected from the group consisting of polyvinyl acetate resin solution, enamel and urethane resin adhesive.

To achieve the above objects, there is provided a method for dyeing the petals of a plant as a method for dyeing the petals of a plant by using a plant petal dyeing composition, comprising a step for filling a plant petal dyeing composition into a container equipped with a underwater pump or an air pump therein; a step for driving the underwater pump or the air pump; and a step for spraying the plant petal dyeing composition onto the petals of the plant by a spray method, thus dyeing the petals of the plant.

To achieve the above objects, there is provided a plant characterized in that the plant is furnished with the petals dyed by means of a dyeing method.

### EFFECTS

In the present invention, it is possible to enhance the added values of products in such a manner that people can have an opportunity to enjoy beauty even in a dark place by dyeing various colors, which cannot be easily found in nature, on various plants such as a real flower or something, or by letting the inherent colors of real flower have noctilucence and photoluminescence effects, and flower petals cannot easily wilt, thus preserving the same for a long time, and any person can easily and quickly dye flower petals regardless of surrounding conditions or environments and can treat the same to have noctilucence and photoluminescence effects while maintaining the inherent natural colors of real flowers.

### BRIEF DESCRIPTION OF THE DRAWINGS

The present invention will become better understood with reference to the accompanying drawings which are given only by way of illustration and thus are not limitative of the present invention, wherein;

FIGS. 1 through 3 are photos which illustrate the roses whose petals are dyed by a method for dyeing petals of a flower according to an embodiment of the present invention; and

FIG. 4 is a flow chart of a method for dyeing petals of a flower according to an embodiment of the present invention.

### MODES FOR CARRYING OUT THE INVENTION

The preferred embodiments of the present invention will be described with reference to the accompanying drawings.

As described earlier, the term “plant” represents various cut-flowers such as rose, chrysanthemum, etc. as well as various plants with petals.

The term “petals” represents leaves or flowers of plants as well as flower buds, etc.

In the present invention, the petals of the plants represent real flowers existing in nature as well as flowers dried and discolored in various drying methods known in the art.

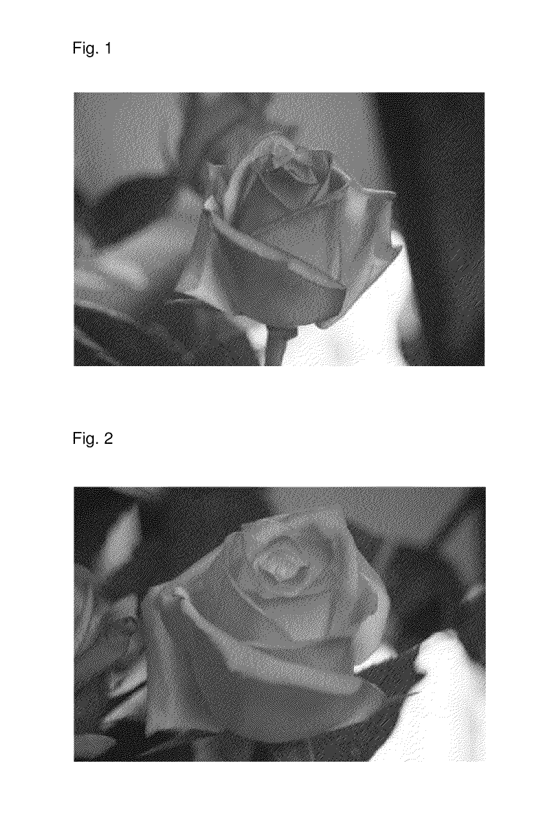

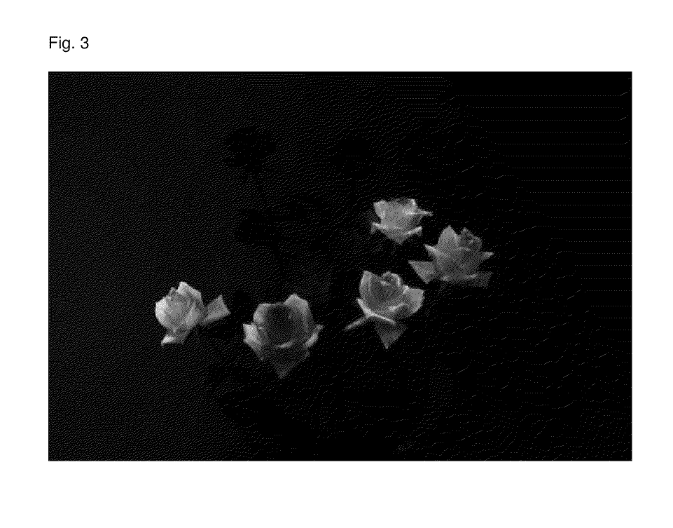

In the present invention, the petals of the roses are applied and dyed in various colors, which are shown in FIGS. 1 to 3 in the forms of images.

It is obvious that since the scope of rights is not limited to the images of FIGS. 1 to 3, the dyed petals of the plants except for the roses belong to the scope of the present invention. In addition, the colors of dyes might be various except for the colors shown in FIGS. 1 to 3.

As shown in the images of FIGS. 1 to 3, it is possible to implement the roses whose colors cannot be found in conventional roses, namely, cannot be found in nature.

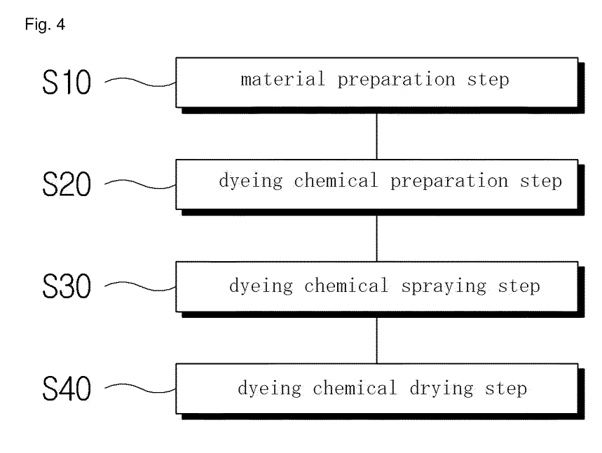

FIG. 4 is a flow chart of a method for dyeing the petals of a plant according to an embodiment of the present invention.

As shown therein, the method for dyeing the petals of a plant according to an embodiment of the present invention comprises a material preparation step S10, a dyeing chemical preparation step S20, a dyeing chemical spraying step S30 and a dyeing chemical drying step S40.

The steps S10 to S40 are sequentially performed, thus implementing a rose with beautiful colors as shown in FIGS. 1 to 3.

As shown in FIGS. 1 to 3, in the material preparation step S10, materials needed for dyeing the petals of a rose are prepared.

The materials to be prepared are oil soluble dyes, and pigment/paint, dissolving substances, and, if needed, adhesive. The adhesive will be described in the descriptions of the dyeing chemical preparation step S20.

The oil soluble dyes will be described in more details. The dyes are classified into an edible coloring agent and a common coloring agent. Since the edible coloring agent is water-soluble, when water is applied to the petals which has been previously sprayed by a dyeing chemical, namely, when it rains or snows, or moisture is applied thereto, the edible coloring agent becomes soluble and flows down together with water.

Since there are not various edible coloring agents, it is almost impossible to obtain a desired color.

In the preferred embodiment of the present invention, the common coloring agent is used as dyes. The common coloring agent generally is classified into a water soluble agent and an oil soluble agent. In the embodiment of the present invention, the oil soluble dyes are preferably applied. A transparent oil soluble dye is more preferably applied among the oil soluble dyes. It is obvious that the scope of the rights of the present invention is not limited such selection.

5 to 20 grams of the oil soluble dyes per 1 to 2 liters of dissolving substances are prepared.

In addition to the above oil soluble dyes, pigment/paint might be further prepared. The pigments and paints might be formed of functional pigment/paint containing chromalusion color pigment/paint, basecoat paint, thermochromic pigment/paint, photochromic pigment/paint, and photoluminescence pigment/paint, and common pigment/paint. Either the functional pigment/paint or the common pigment/paint might be selected.

It is preferred that 10 to 110 grams of pigment/paint per 1 to 2 liters of dissolving substance are prepared.

Here, the dissolving substance is used to dissolve and mix the oil soluble dye and the pigment/paint. Since it is hard to mix the oil soluble dye and the pigment/paint and directly to spray on the petals, the dissolving substance is generally used as a means for dissolving or mixing the oil soluble dye and the pigment/paint.

The dissolving substance might be selected from the group consisting of alcohol, acetone and MEK each having relatively stronger evaporation performances. The amount of the dissolving substance increases or decreases in proportion to the amounts of the oil soluble dye and the pigment/paint.

When the amount of the oil soluble dye increases regardless of the mixing ratio, precipitation occurs, which results in causing problematic operation with sprayer. Since over coloring petal phenomenon occurs, dyes might smear the hands.

On the contrary, when the adding ratio of the alcohol is more than the above ration, the color might become thin in the course of coloring, thus making the expression of a desired color impossible. It is preferred to follow the above mixing ratios. It is further preferred that 1.5 liters of alcohol per 10 grams of dyes is added, not limiting thereto.

When alcohol is used as a dissolving substance, the oil soluble dye and pigment/paint are mixed with alcohol and become soluble. When the dissolved dyes are sprayed onto the petals, alcohol begins to evaporate as soon as dyes touch the petals, so desired color, namely, oil soluble dyes and pigment/paint can be colored on the petals.

In particular, since oil soluble dyes do not dissolve in water but dissolve in oil, even when the oil soluble dyes smear the hands, the color does not blur. Even when hands touch the dyes, the dyes do not smear the hands.

In the dyeing chemical preparation step S20, 1 to 2 liters of dissolving substance, 5 to 20 grams of oil soluble transparent dyes and 10 to 110 grams of pigment/paint are dissolved or mixed at the above ratios.

In the course of the dissolution or mixing, a tank might be further used. An agitator might be preferably used for smooth dissolution or mixing.

In the dyeing chemical step S20, a certain adhesive might be used.

In case that the adhesive is mixed, the adhesive might be selected from the group consisting of polyvinyl acetate solution, enamel and urethane resin adhesives, which all can be strongly adhered with the solution having good evaporation performance. It is preferred that it is added at a weight ratio of 10 to 20 with respect to the 100 weight of the solution in which oil soluble dyes, pigment/paint and dissolving substances are mixed in the course of the dyeing chemical preparation step S20.

In the dyeing chemical spraying step S30, the dyes mixed at the above ratios are subject to being sprayed onto the petals using a certain sprayer.

The sprayer might be a spray gun or an air brush each connected with a compressor.

The dyes prepared in the dyeing chemical preparation step S20 is filled in the spray gun or the air brush connected with the compressor. With the pneumatic pressure of the compressor being increased, the dyes are sprayed onto the petals using the spray gun or the air brush. When the dyes touch the petals, the alcohol evaporates, and the oil soluble dyes and pigment/paint dissolved in the alcohol are coated on the petals.

Finally, in the dyeing chemical drying step S40, the dyes are subject to natural drying, which process might be omitted.

When it is needed to fast dry the dyes, the dyeing chemical spraying step S30 is performed, and then a corresponding plant is forced into an additional dryer, thus forcibly drying.

In case that the above method is used, it is possible to significantly increase the added values of the products by coloring various colors, which cannot be found in nature, on various plants such as real flowers, etc., thus creating a new beauty in plants.

The present invention makes it possible to prevent the petals from being wilted and dirty and to prevent the dyes from smearing the hands. It is possible for anyone to easily and fast dye the petals regardless of the surrounding situations and environments.

For reference, in case of the rose which has the most expensive royalty, most of rose farmers are forced to change the variety of the rose at every two or three years, but the present invention makes it possible to express a wide color range of roses by using one kind of the rose, thus helping enhance the income of the farmers, who are suffering from economical depressions, while significantly saving the royalties which are to be paid overseas.

According to the present invention, it is possible to enhance the added values of products in such a manner that people can have an opportunity to enjoy beauty even in a dark place by dyeing various colors, which cannot be easily found in nature, on various plants such as a real flower or something, or by letting the inherent colors of real flower have noctilucence and photoluminescence effects, and flower petals cannot easily wilt, thus preserving the same for a long time, and any person can easily and quickly dye flower petals regardless of surrounding conditions or environments and can treat the same to have noctilucence and photoluminescence effects while maintaining the inherent natural colors of real flowers.

Various embodiments for dyeing the petals of roses using the method of the present invention will be described.

### First Embodiment

1 to 2 liters of alcohol, 5 to 20 grams of oil soluble dyes, 10 to 50 grams of chromalusion color pigment/paint and a polyvinyl acetate solution adhesive (at least one adhesive was selected from the group consisting of polyvinyl acetate solution, enamel and urethane resin adhesive) each were prepared by certain amounts, respectively.

After the above materials were prepared, 5 to 20 grams of oil soluble dyes and 10 to 50 grams of chromalusion color pigment/paint per 1 to 2 liters of alcohol were mixed, and the oil soluble dyes and the chromalusion color pigment/paint were dissolved or mixed using the alcohol. The polyvinyl acetate resin solution adhesive was mixed at a ratio of 10 weight % to 20 weight % with respect to 100 weight % of the mixed solution, thus preparing dyeing chemicals.

The prepared dyeing chemical was inputted into a spray gun with a container having a small size underwater pump by a certain amount, and the spray gun was connected with the compressor, and the compressor was turned on while maintaining an air pressure of about 50 kg/cm². The underwater pump in the container with the dyeing chemicals was activated, and the front end of the nozzle of the spray gun was oriented toward the petals to be coated, and the dyeing chemicals were sprayed onto the petals of the rose with the aid of the compressed air. The coated petals were dried at room temperature following the spraying, thus substantially evaporating alcohol components.

As a result, as shown in FIGS. 1 to 3, the present invention made it possible to express beautiful colors at day time which colors could not be expressed in common roses, and various colors and glittering pearl colors like chameleon colors were obtained when viewing with sunshine being at a backside.

### Second Embodiment

1 to 2 liters of alcohol, 5 to 20 grams of oil soluble dyes, 10 to 50 grams of basecoat paint and 50 to 110 grams of photoluminescence pigment were prepared.

After the above materials were prepared, 5 to 20 grams of oil soluble dyes and 10 to 50 grams of basecoat pigment per 1 to 2 liters of alcohol were mixed, and the oil soluble dyes and the basecoat pigment were dissolved or mixed using the alcohol. 50 to 110 grams of photoluminescence pigment were further mixed with 1 liter of the mixed solution, thus preparing dyeing chemical.

The prepared dyeing chemical was inputted into a spray gun with a container having a small size underwater pump by a certain amount, and the spray gun was connected with the compressor, and the compressor was turned on while maintaining an air pressure of about 50 kg/cm². The underwater pump in the container with the dyeing chemicals was activated, and the front end of the nozzle of the spray gun was oriented toward the petals to be coated, and the dyeing chemicals were sprayed onto the petals of the rose with the aid of the compressed air. The coated petals were dried at room temperature following the spraying, thus substantially evaporating alcohol components.

As a result, the present invention made it possible to express beautiful colors at day time which colors could not be expressed in common roses, and glittering pearl colors were observed when viewing with sunshine being at a backside, and shining was observed at night time with the aid of photoluminescence pigment.

### Third Embodiment

1 to 2 liters of alcohol, 5 to 20 grams of oil soluble dyes, 10 to 50 grams of thermochromic pigment, 50 to 110 grams of photoluminescence pigment, and a certain amount of polyvinyl acetate resin solution adhesive were prepared, respectively.

After the above materials were prepared, 5 to 20 grams of oil soluble dyes and 10 to 50 grams of thermochromic pigment per 1 to 2 liters of alcohol were mixed, and the oil soluble dyes and the thermochromic pigment were dissolved or mixed using the alcohol. 50 to 110 grams of photoluminescence pigment were further mixed with 1 liter of the mixed solution, thus preparing dyeing chemical by mixing a polyvinyl acetate resin solution adhesive at a ratio of 10 to 20 weight % with respect to 100 weight % of the mixed solution.

The prepared dyeing chemical was inputted into a spray gun with a container having a small size underwater pump by a certain amount, and the spray gun was connected with the compressor, and the compressor was turned on while maintaining an air pressure of about 50 kg/cm². The underwater pump in the container with the dyeing chemicals was activated, and the front end of the nozzle of the spray gun was oriented toward the petals to be coated, and the dyeing chemicals were sprayed onto the petals of the rose with the aid of the compressed air. The coated petals were dried at room temperature following the spraying, thus substantially evaporating alcohol components.

As a result, the present invention made it possible to express beautiful colors at day time which colors could not be expressed in common roses, and the colors were changed depending temperatures, and shining was observed at night time with the aid of photoluminescence pigment.

### Fourth Embodiment

1 to 2 liters of alcohol, 5 to 20 grams of oil soluble dyes, 10 to 50 grams of photochromic pigment, 50 to 110 grams of photoluminescence pigment, and a certain amount of polyvinyl acetate resin solution adhesive were prepared, respectively.

After the above materials were prepared, 5 to 20 grams of oil soluble dyes and 10 to 50 grams of photochromic pigment per 1 to 2 liters of alcohol were mixed, and the oil soluble dyes and the photochromic pigment were dissolved or mixed using the alcohol. 50 to 110 grams of photoluminescence pigment were further mixed with 1 liter of the mixed solution, thus preparing dyeing chemical by mixing a polyvinyl acetate resin solution adhesive at a ratio of 10 to 20 weight % with respect to 100 weight % of the mixed solution.

The prepared dyeing chemical was inputted into a spray gun with a container having a small size underwater pump by a certain amount, and the spray gun was connected with the compressor, and the compressor was turned on while maintaining an air pressure of about 50 kg/cm². The underwater pump in the container with the dyeing chemicals was activated, and the front end of the nozzle of the spray gun was oriented toward the petals to be coated, and the dyeing chemicals were sprayed onto the petals of the rose with the aid of the compressed air. The coated petals were dried at room temperature following the spraying, thus substantially evaporating alcohol components.

As a result, the present invention made it possible to express beautiful colors at day time which colors could not be expressed in common roses, and the colors were changed depending violet ray of sunshine, and shining was observed at night time with the aid of photoluminescence pigment.

### Fifth Embodiment

1 to 2 liters of alcohol, 5 to 20 grams of oil soluble dyes, 10 to 50 grams of thermochromic pigment, 10 to 50 grams of photochromic pigment, 50 to 110 grams of photoluminescence pigment, and a certain amount of polyvinyl acetate resin solution adhesive were prepared, respectively.

After the above materials were prepared, 5 to 20 grams of oil soluble dyes, 10 to 50 grams of thermochromic pigment, and 10 to 50 grams of photochromic pigment per 1 to 2 liters of alcohol were mixed, and the oil soluble dyes, the thermochromic pigment and the photochromic pigment were dissolved or mixed using the alcohol. 50 to 110 grams of photoluminescence pigment were further mixed with 1 liter of the mixed solution, thus preparing dyeing chemical by mixing a polyvinyl acetate resin solution adhesive at a ratio of 10 to 20 weight % with respect to 100 weight % of the mixed solution.

The prepared dyeing chemical was inputted into a spray gun with a container having a small size underwater pump by a certain amount, and the spray gun was connected with the compressor, and the compressor was turned on while maintaining an air pressure of about 50 kg/cm². The underwater pump in the container with the dyeing chemicals was activated, and the front end of the nozzle of the spray gun was oriented toward the petals to be coated, and the dyeing chemicals were sprayed onto the petals of the rose with the aid of the compressed air. The coated petals were dried at room temperature following the spraying, thus substantially evaporating alcohol components.

As a result, the present invention made it possible to express beautiful colors at day time which colors could not be expressed in common roses, and the colors were changed depending violet ray of sunshine, and the colors were changed depending on surrounding temperature as well, and shining was observed at night time with the aid of photoluminescence pigment.

As the present invention may be embodied in several forms without departing from the spirit or essential characteristics thereof, it should also be understood that the above-described examples are not limited by any of the details of the foregoing description, unless otherwise specified, but rather should be construed broadly within its spirit and scope as defined in the appended claims, and therefore all changes and modifications that fall within the meets and bounds of the claims, or equivalences of such meets and bounds are therefore intended to be embraced by the appended claims.

1. A method for dyeing the petals of plant, comprising:
a material preparation step for preparing oil soluble dye as dyeing material, pigment/pant and a dissolving substance for dissolving or mixing the oil soluble dye and the pigment/paint;
a dyeing chemical preparation step for preparing a dyeing chemical by dissolving or mixing the oil soluble dye and the pigment/paint which have been prepared at a certain mixing ratio;
a dyeing chemical spraying step for filling the dyeing chemical into a certain sprayer and spraying the dyeing chemical onto the petals of a plant via the sprayer; and
a dyeing chemical drying step for drying the dyeing chemical following the dyeing chemical spraying step.

2. The method of claim 1, wherein said oil soluble dye is selected from common dyes except for edible dyes, and said pigment/paint is at least one selected from the group consisting of a functional pigment/paint containing chromalusion color pigment/paint, basecoat paint, thermochromic pigment/paint, photochromic pigment/paint, and photoluminescence pigment/paint, and a common pigment/paint, and said dissolving substance is selected from the group consisting of alcohol, acetone, and MEK which all have high evaporation performance.

3. The method of claim 1, wherein said certain mixing ratio is 5 to 20 grams per 1 to 2 liters of the dissolving substance in case of the oil soluble transparent dye, and is 10 to 110 grams in case of the pigment/paint.

4. The method of claim 1, wherein said dyeing chemical preparing step includes a sub-step for further adding a certain adhesive.

5. The method of claim 4, wherein said adhesive is one selected from the group consisting of polyvinyl acetate resin solution, enamel and urethane resin adhesive, and said adhesive is mixed at a ratio of 10 to 20 weight % with respect to 100 weight % of the solution mixed with the oil soluble dye, the pigment/paint and the dissolving substance in the course of preparing dyeing chemicals.

6. A plant characterized in that said plant is furnished with the petals dyed by the method of dyeing cited in claim 1.

7. A plant petal dyeing composition characterized in that said composition is prepared by dissolving or mixing 5 to 20 grams of oil soluble transparent dye and 10 to 110 grams of pigment/paint per 1 to 2 liters of the dissolving substance as a composition for dyeing the petals of plant.

8. The composition of claim 7, wherein said oil soluble dye is selected from common dyes except for edible dyes, and said pigment/paint is at least one selected from the group consisting of a functional pigment/paint containing chromalusion color pigment/paint, basecoat paint, thermochromic pigment/paint, photochromic pigment/paint, and photoluminescence pigment/paint, and a common pigment/paint, and said dissolving substance is selected from the group consisting of alcohol, acetone, and MEK which all have high evaporation performance.

9. The composition of claim 7, further comprising:
an adhesive which is mixed at a ratio of 10 to 20 weight % with respect to 100 weight of the solution mixed with the oil soluble dye, the pigment/paint and the dissolving substance, and said adhesive is at least one selected from the group consisting of polyvinyl acetate resin solution, enamel and urethane resin adhesive.

10. A method for dyeing the petals of a plant as a method for dyeing the petals of a plant by using a plant petal dyeing composition of claim 7, comprising:
a step for filling a plant petal dyeing composition into a container equipped with an underwater pump or an air pump therein;
a step for driving the underwater pump or the air pump; and
a step for spraying the plant petal dyeing composition onto the petals of the plant by a spray method, thus dyeing the petals of the plant.

11. A plant characterized in that said plant is furnished with the petals dyed by means of a dyeing method of claim 7.

12. A plant characterized in that said plant is furnished with the petals dyed by the method of dyeing cited in claim 2.

13. A plant characterized in that said plant is furnished with the petals dyed by the method of dyeing cited in claim 3.

14. A plant characterized in that said plant is furnished with the petals dyed by the method of dyeing cited in claim 4.

15. A plant characterized in that said plant is furnished with the petals dyed by the method of dyeing cited in claim 5.

16. A plant characterized in that said plant is furnished with the petals dyed by means of a dyeing method of claim 8.

17. A plant characterized in that said plant is furnished with the petals dyed by means of a dyeing method of claim 9.

18. A plant characterized in that said plant is furnished with the petals dyed by means of a dyeing method of claim 10.
